(19) United States
(12) Patent Application Publication
(10) Pub. No.: US 2024/0122163 A1
(43) Pub. Date: Apr. 18, 2024

(54) A METHOD FOR CONSTRUCTING A COFILIN-1 TRANSGENIC MODEL AND USE THEREOF

(71) Applicant: National Yang Ming Chiao Tung University, Taipei (TW)

(72) Inventors: Yi-Jang LEE, Taipei City (TW); Yu-Chuan LIN, Taipei City (TW); Min-Ying LIN, Taipei City (TW); Bing-Ze LIN, Taipei City (TW); Chia-Yun KANG, Taipei City (TW)

(21) Appl. No.: 18/165,038

(22) Filed: Feb. 6, 2023

(30) Foreign Application Priority Data
Oct. 13, 2022 (TW) 111138901

(51) Int. Cl. A01K 67/027

(52) U.S. Cl. CPC A01K 67/0275; A01K 2217/05; A01K 2227/105; A01K 2267/02; A01K 2267/0393; C07K 2319/60

(57) ABSTRACT

The present invention demonstrated a Cre-loxP based cofilin-1 transgenic animal model to address the pathophysiological role of over-expressed cofilin-1 on systemic development.

### CROSS-REFERENCE TO RELATED APPLICATIONS

This Non-provisional application claims priority under 35 U.S.C. § 119(a) on Patent Application No(s). 111138901 filed in Taiwan, Republic of China on Oct. 13, 2022, the entire contents of which are hereby incorporated by reference.

### REFERENCE TO AN ELECTRONIC SEQUENCE LISTING

The content of the electronic sequence listing (YC-22PA073-US-(SequenceListing).xml; Size: 11,696 bytes; and Date of Creation: Feb. 6, 2023) is herein incorporated by reference in its entirety.

### FIELD OF THE INVENTION

The present invention relates to construct an over-expressed cofilin-1 transgenic model for exploring the pathophysiological role of systemic development.

### BACKGROUND OF THE INVENTION

Aging is an important risk factor for multiple human diseases including cognitive impairment, cancer, arthritis, vision loss, osteoporosis, diabetes, cardiovascular disease, and stroke. In addition to normal synapse loss during natural aging, synapse loss is an early pathological event common to many neurodegenerative conditions and is the best correlate to the neuronal and cognitive impairment associated with these conditions. As such, aging remains the single most dominant risk factor for dementia-related neurodegenerative diseases such as Alzheimer's disease (AD) (Bishop, N. A. et al., Neural mechanisms of ageing and cognitive decline. Nature 464(7288), 529-535 (2010); Heeden, T. et al., Insights into the ageing mind: a view from cognitive neuroscience. Nat. Rev. Neurosci. 5(2), 87-96 (2004); Mattson, M. P., et al., Ageing and neuronal vulnerability. Nat. Rev. Neurosci. 7(4), 278-294 (2006)). Aging affects all tissues and functions of the body including the central nervous system, and a decline in functions such as cognition and motor activity can severely impact quality of life.

The actin depolymerizing factor (ADF)/cofilin family encodes ˜19 kD actin-binding proteins in mammals and includes cofilin-1, cofilin-2, and ADF (Bernstein B W, Bamburg J R., ADF/cofilin: a functional node in cell biology. Trends Cell Biol. Apr.; 20(4):187-95 (2010)). Cofilin-1 and ADF are co-expressed in non-muscle cells, although cofilin-1 is usually predominantly expressed in various cell types (Hotulainen, P. et al., Actin-depolymerizing factor and cofilin-1 play overlapping roles in promoting rapid F-actin depolymerization in mammalian nonmuscle cells. Mol Biol Cell. Feb.; 16(2):649-64. (2005)).

Cofilin-1 (CFL-1) is one of actin-binding proteins. The main function of CFL-1 is to regulate the depolymerization of actin filaments. It also affects the shape, migration, division and other functions of the cell. In previous studies, it was found that overexpression of CFL-1 would cause cell swelling, and senescence, etc.

However, most of the existing aged animal models are used by cofilin gene knockout to explore the role of Cofilin in organisms. The functional research on the overexpression of Cofilin is focus on the biochemical studies or in vitro. Until now transgenic cofilin multicellular organisms are not developed.

Therefore, the model of the inducible expression of the transgenic cofilin gene technology and its application in the anti-aging drug screening platform are the problems that need to be solved urgently in this field.

### SUMMARY OF THE INVENTION

An object of the invention is to develop a new multicellular organism method of inducible expression of the transgenic cofilin gene technology.

Genetically engineered mouse models are commonly preferred for studying the human disease due to genetic and pathophysiological similarities between mice and humans. In particular, the Cre/lox site-specific recombination system has emerged as an important tool for the generation of conditional somatic mouse mutants. A major technical advance in terms of in vivo inducibility was the development of ligand-dependent Cre recombinases that can be activated by administration of tamoxifen to the animal (Feil, S. et al., Inducible Cre mice. Methods Mol Biol. 530:343-63. (2009)).

Temporal control of cre activation can be further increased beyond that of the promoter used by the inclusion of an inducible cre protein such as the tamoxifen responsive cre/ERT2 fusion protein. This inducible system has cre fused to a mutated estrogen receptor (ERT2) such that Cre/ERT2 is normally sequestered in the cytoplasm and inactive, but, when tamoxifen binds ERT2, the cre fusion protein translocates to the nucleus, where it is active. Therefore, the expression pattern of cre from the inducible system reports on a combination of the promoter driving cre, but only during when tamoxifen is present (Korecki, A. J. et al., Twenty-Seven Tamoxifen-Inducible iCre-Driver Mouse Strains for Eye and Brain, Including Seventeen Carrying a New Inducible-First Constitutive-Ready Allele. Genetics. Apr.; 211(4):1155-1177. (2019)).

In contrast to the inducible allele, constitutive cre reflects only the promoter used. Cre is always present in the nucleus, and thus is a reporter of any expression from development through adulthood.

Here this present invention describes how tamoxifen-dependent Cre recombinases, so-called Cre-ERT2 recombinases, work and how they can be used to generate time- and tissue-specific mouse mutants. The present invention will give an overview of available Cre-ERT2 transgenic mouse lines and present protocols that detail the generation of experimental mice for inducible transgene studies, the induction of recombination by tamoxifen treatment, and the analysis of the quality and quantity of recombination by reporter gene and target gene studies (Feil S et al., Inducible Cre mice. Methods Mol Biol. 530:343-63. (2009)).

Detailed description of the invention is given in the following embodiments with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee

The foregoing summary, as well as the following detailed description of the invention, will be better understood when reading in conjunction with the appended drawings.

FIG. 1A depicts the genotyping results of the transgenic founder mice by polymerase chain reaction (PCR). The symbols of asterisk represent that 12 founder mice were successfully bearded transgene integration by using PCR screening.

FIG. 1B depicts that the founder mice were visualized under UV. It clearly showed that these mice expressed GFP protein for visualizing the fluorescence comparing to wild-type (WT) mice.

FIG. 2A depicts that the mating strategy using conventional Cre-ERT2 transgenic mice for conditional pop-out of the loxP-emGFP-loxP cassette by giving Tamoxifen.

FIG. 2B depicts that 3 of mice carry the Cre-ERT2 cassette after initial genotyping. P: Cre plasmid DNA, R: red.

FIG. 2C depicts that these five mice were examined the red fluorescence emitted from the excised ear tissues.

FIG. 3A depicts genotyping for Cre fragment (560 bp), loxP fragment (423 bp) and endogenous β-actin intron1 fragment (320 bp) from corresponding numbered mice.

FIG. 3B depicts that emGFP pop out confirmation (emergence of 247 bp DNA fragment) from transgenic mice fed with tamoxifen. M: 100 bp marker. B: Genomic DNA before loxP-emGFP pop-out.

FIG. 3C depicts that a PCR product (500 bp) of transgenic 3XFlag-cofilin-1 from each loxP-emGFP-loxP/Cre-ERT2 transgenic mouse. P1 and P2 represent positive control of Cfl1 cDNA on plasmid for 10× and 20× dilution.

In FIGS. 4, A and B depict that the GFP fluorescence was reduced after TAM treatment in transgenic mouse qualitatively and FIG. 4C depicts this information quantitatively. Data are expressed as means±SEM.

FIG. 4D depicts that the expression of cofilin-1 mRNA up-regulated about 5 folds for transgenic mice compared to WT mice. Data are expressed as means±SEM. **: p<0.01.

In FIG. 5, A-D depict that cofiilin-1 protein was over-expressed in ear tissue of transgenic mouse treated with TAM compared to that of WT mouse. Scale: 50 lam.

FIG. 6 depict that the IHC staining was quantified by calculating the IHC score [proportion (1-4)×intensity (0-3)]. Data are expressed as means±SEM.

FIG. 7 depict that the change of body weight in cofilin-1 transgenic mouse fed with TAM feed.

FIG. 8 depict that the movement of young mouse at night was frequently recorded compared to that of old mouse and cofilin-1 transgenic mouse. Data are expressed as means±SEM. *: p<0.01.

In FIG. 9, A-D depict that the lateral ventricle of cofilin-1 transgenic mice (Tg-CFL1) was larger than that of wild-type mouse at the similar slice of transverse images. A and B represent WT mouse. C and D represent transgenic mouse.

In FIG. 10, A-D depict that the 3D reconstruction of coronal imaging was performed to compare the difference between the lateral ventricles of wild-type mice and cofilin-1 transgenic mice. A represents WT #1 (21 weeks), B represents WT #2 (19 weeks), C represents Tg-CFL1 #1 (21 weeks), and D represents Tg-CFL1 #2 (19 week).

### DETAILED DESCRIPTION OF THE INVENTION

While preferred embodiments of the invention are shown and described herein, such embodiments are provided by way of example only and are not intended to limit the scope of the invention otherwise. Various alternatives to the described embodiments of the invention may be employed in practicing the invention.

Unless otherwise defined, all technical terms used herein have the same meaning as commonly understood by one of ordinary skill in the art to which this invention belongs.

A subject may be a non-human multicellular organism, such as cat, dog, rabbit, cow, cattle, horse, pig, sheep, goat, monkey, guinea pig, gerbil, rat, mouse, zebrafish, Caenorhabditis elegans, fruit flies, etc.

### EXAMPLES

The embodiments encompassed herein are now described with reference to the following examples. These examples are provided for the purpose of illustration only, and the disclosure encompassed herein should in no way be construed as being limited to these examples, but rather should be construed to encompass any and all variations which become evident as a result of the teachings provided herein.

Each treatment condition was performed in at least triplicate in an experiment. For in vivo assays, mouse numbers used in each group were indicated. Data were presented as means±SD. For data collected from at least three independent experiments, the statistical results were shown as means ±SEM. For representative images, at least three independent experiments showed similar results. For comparison of groups, the Student's t-test or one-way ANOVA was used. *<0.05.

### Example 1

Establishment of Cofilin-1 Transgenic Mice

This animal model is a “STOP-n-GO” system that contain an emGFP fluorescent protein reporter gene that can allow an easy prescreening of putative cofilin-1 transgene mice. The construct backbone is derived from the Bacterial Artificial Chromosome clone (BAC clone) harboring a murine full β-actin gene. The loxp-emGFP-BGHpA-NeoR-loxP cassette and 3×Flag-Cfl1-BGHpA cassette were inserted into intron 1 and exon 2 of β-actin gene to establish a conditional transgene construct, respectively.

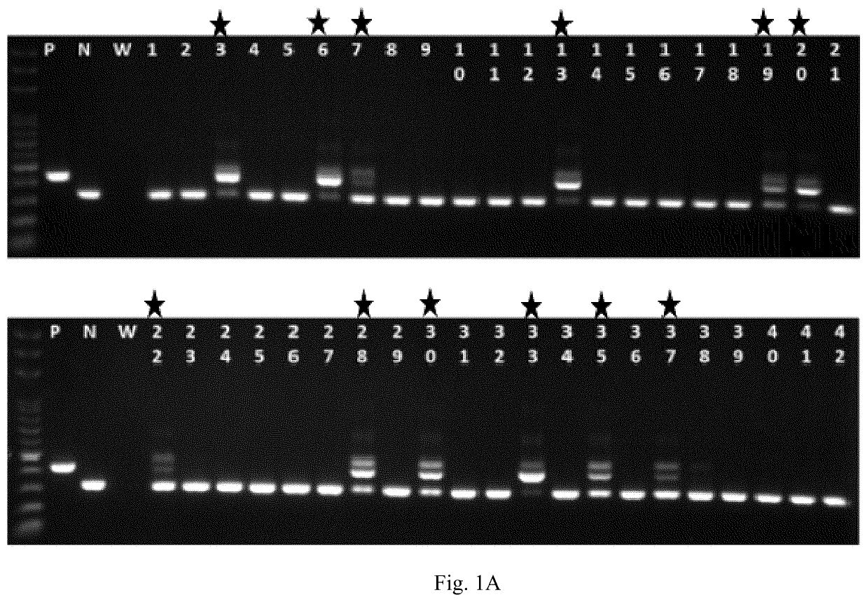

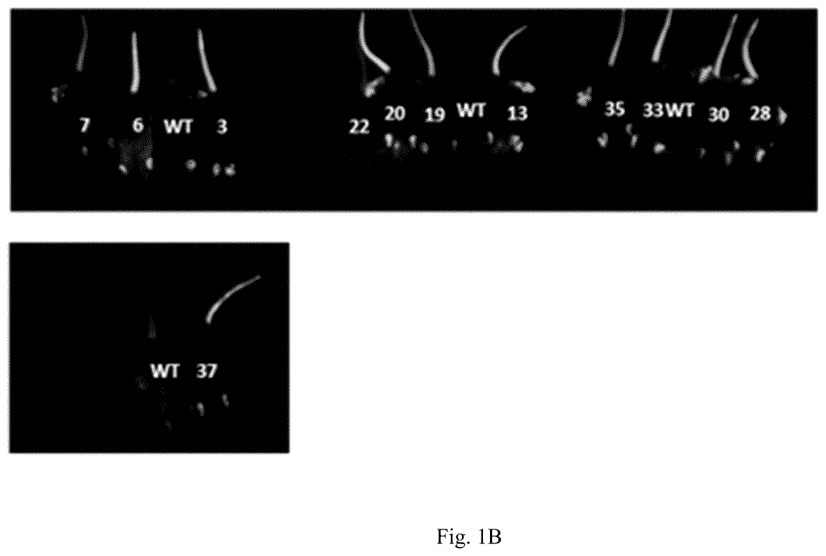

The construct was microinjected to the embryos to produce the conditional transgenic C57BL/6 mice with whole-body expression of GFP reporter gene. It is expected that the BAC clone would process homologous recombination and place the cassettes mentioned above. After genotyping of the transgenic founder mice by polymerase chain reaction (PCR) of genomic DNA extracted from tails, the results showed that 12 founder mice were successfully screened (FIG. 1A). The founder mice were visualized under UV. It clearly showed that these mice expressed GFP protein for visualizing the fluorescence comparing to wild-type (WT) mice (FIG. 1B). These founder mice were categorized as 1st FO (genotype: loxP-emGFP-loxP-Cfl1) and 2nd FO (genotype: Cre-ERT2).

Two mice were picked with strongest PCR bands shown on the agarose gel (#20, #33) for mating with wild-type C57BL/6 mice according to the genotyping results (FIG. 1A and FIG. 1B). The above-mentioned results were expected to obtain the next generation with stable expression of transgenic cfl1 controlled by loxP-emGFP-loxP locus. The present invention was subsequently obtained the loxP-Cfl1/+batch of newborn mice from #20 founder mouse as confirmed by PCR based genotyping. These mice will be used for mating with another Cre-ERT2 transgenic mice for conditional expression of cofilin-1 in transgene mice.

### Example 2

Characterization of Cre-ERT2 Transgenic Mouse

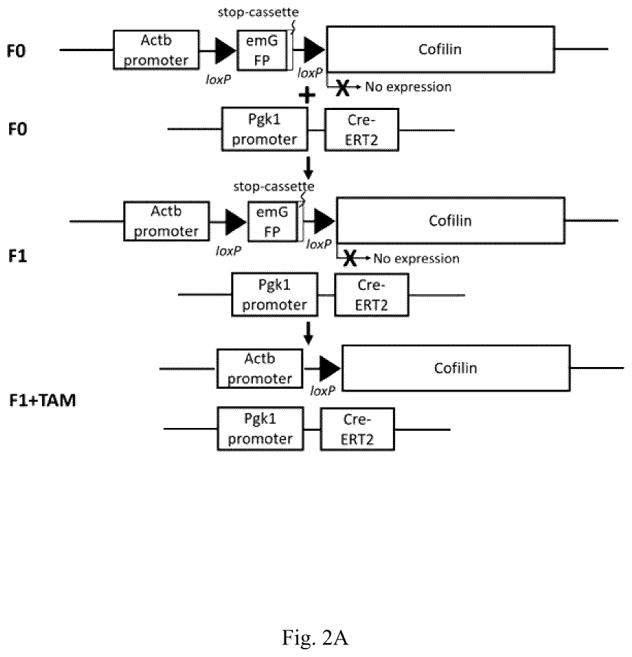

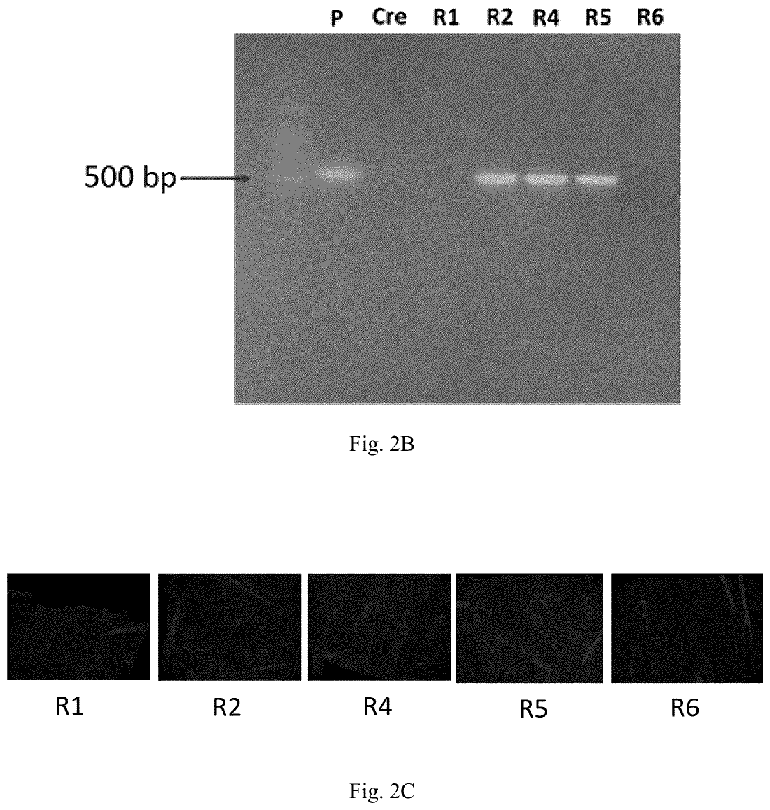

The conditional cofilin-1 transgenic mice, named loxP-emGFP-loxP-Cfl1 mice, can only express exogenous cofilin-1 by mating with Cre transgenic mice. The mating strategy using conventional Cre-ERT2 transgenic mice for conditional pop-out of the loxP-emGFP-loxP cassette by giving Tamoxifen was designed in this embodiment (FIG. 2A). The full name and construct of Cre-ERT2 mouse is C57BL/6-Tg(Pgk1-RFP,-cre/ERT2)3NarI, from National Animal Research Labs Taiwan.

Five mice were delivered from that Labs, and 3 of them carry the Cre-ERT2 cassette after initial genotyping (FIG. 2B). These five mice also were examined the red fluorescence emitted from the excised ear tissues as these transgenic mice bring red fluorescence protein (RFP) genes. The result of fluorescent visualization was consistent with that of genotyping, i.e., stronger fluorescent signals were visualized in those harboring Cre-ERT2 cassettes (FIG. 2C)

### Example 3

Genotyping of the Offspring Mated by loxP-emGFP-IoxP-Cfl1 Mouse and Cre-ERT2 Mouse

After offspring was bred from two parent transgenic mice mentioned above, the expression of green fluorescence could be quickly observed from the litters. The green litters were further investigated after weaning, and the expression of green fluorescence could be directly detected in eyeballs. Subsequently, the foot fingers of each candidate mouse were cut and added with 100 ml DirectPCR lysis reagent in individual microcentrifugation tube. Protein kinase K (0.4 mg/mL) was mixed with the sample by vortex and incubated at 55° C. overnight. The kinase reaction was inactivated by 85° C. for 45 minutes followed by centrifugation at 8,000 rpm for 2 minutes. The supernatant containing genomic DNA was transferred to a clean microcentrifugation tube for storage under −20° C.

For genotyping of Cre and loxP co-existed in fluorescent offspring, the Multiplex PCR was used to simultaneously amplify these fragments in one reaction. The primer set of Cre fragment is Forward: 5′-CTAAACATGCTTCATCGTCGGTC-3′ (SEQ ID NO: 1), and Reverse: 5′-TCTGACCAGAGTCATCCTTAGCG-3′ (SEQ ID NO: 2); that of loxP fragment is Forward: 5′-CCGAAAGTTGCCTTTTATGGCTC-3′ (SEQ ID NO: 3), and Reverse: 5′-GCTGCAAAGAGTCTACACGCTAGG-3′ (SEQ ID NO: 4). The reaction condition is as described below.

TABLE 1
The reaction condition of polymerase chain reaction.

| | Temperature | | |
|---|---|---|---|
| Step | (° C.) | Time (min) | Number of cycles |
| 1 | 94 | 3 | 1 |
| 2 | 94 | 0.5 | 35 |
| 3 | 55 or 57 | 0.5 | 35 |
| 4 | 72 | 0.8 | 35 |
| 5 | 72 | 3 | 1 |

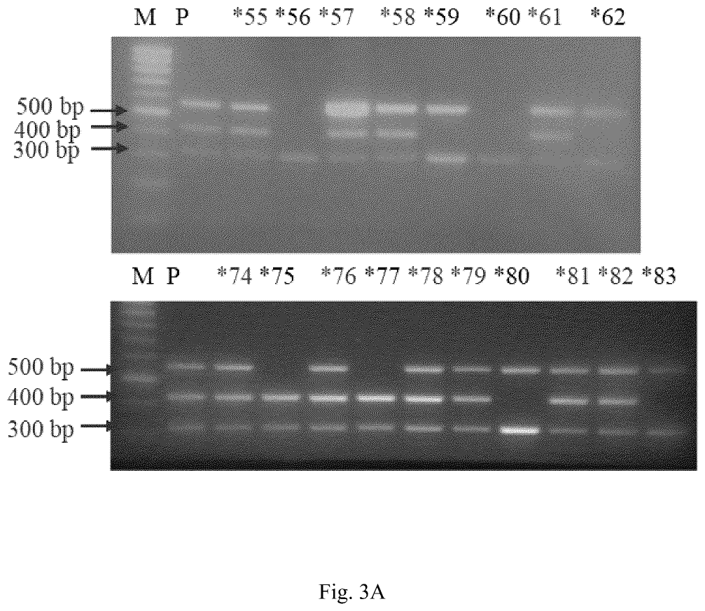

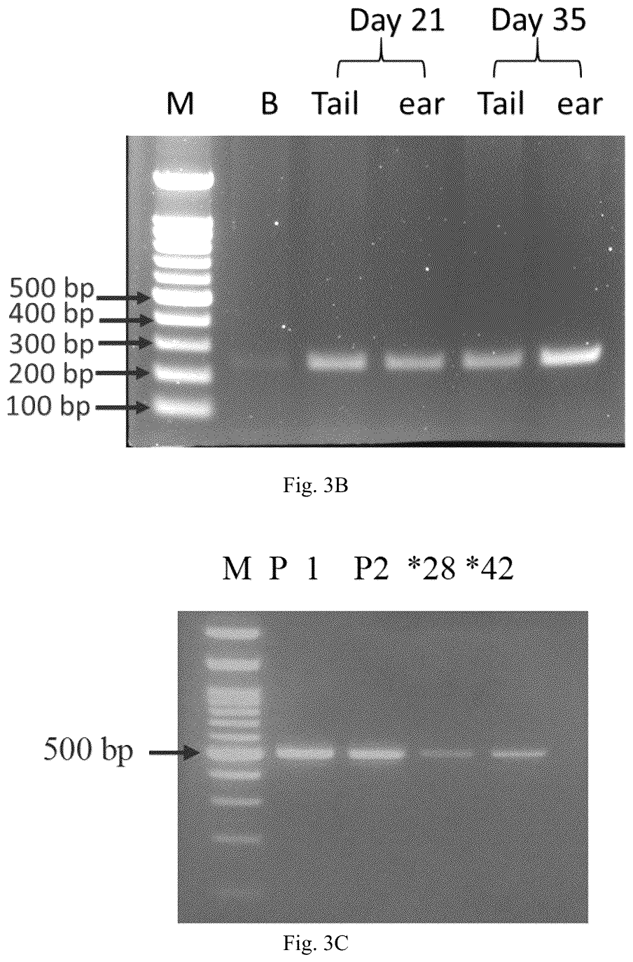

The result showed that three bands shown on the agarose gel were 560 bp (Cre fragment), 423 bp (loxP fragment) and 320 bp (endogenous (3-actin intron1 fragment) could be visualized in positive control and red marked genomic samples from corresponding numbered mice (FIG. 3A). Therefore, not all fluorescent mice harbor both Cre and loxP fragments. Further, 2 mice were randomly selected with expected genotypes to verify the pop-out of loxP-emGFP-loxP cassette using another set of primers that correspond to the position flanked the cassette. The sequence of this set is: Actb5UF4: 5′-GCTGTGGCGTCCTATAAAACC-3′ (SEQ ID NO: 7) and ActbloxPB4: 5′-GACCCTGCAGTGAGGATAACTTC-3′ (SEQ ID NO: 8). After transgnenic mice treated with tamoxifen, a 247 hp fragment will be amplified by genomic PCR compared to untreated control. It showed that transgenic mice fed with tamoxifen for 21 days was sufficient to pop put the loxP-emGFP-loxP cassette (FIG. 3B). It was confirmed that the 3XFlag-cofilin-1 cassette was also existed in the transgenic mice using the cofilin-1 specific primers: Forward: 5′-ATGGCCTCCGGTGTGTGGCTGTC-3 ‘ (SEQ ID NO: 5) and Reverse: 5 TCACAAAGGCTTGCCCTCC-3’ (SEQ ID NO: 6). A 500 bp PCR product can be amplified from the genomic DNA of each loxP-emGFP-loxP/Cre-ERT2 transgenic mouse (FIG. 3C). These results demonstrated that a conditioned cofilin-1 transgenic mouse model was successfully established using current strategy.

### Example 4

Evaluation of Cofilin-1 Expression in loxP-emGFP-loxP/Cre-ERT2 Transgenic Mouse

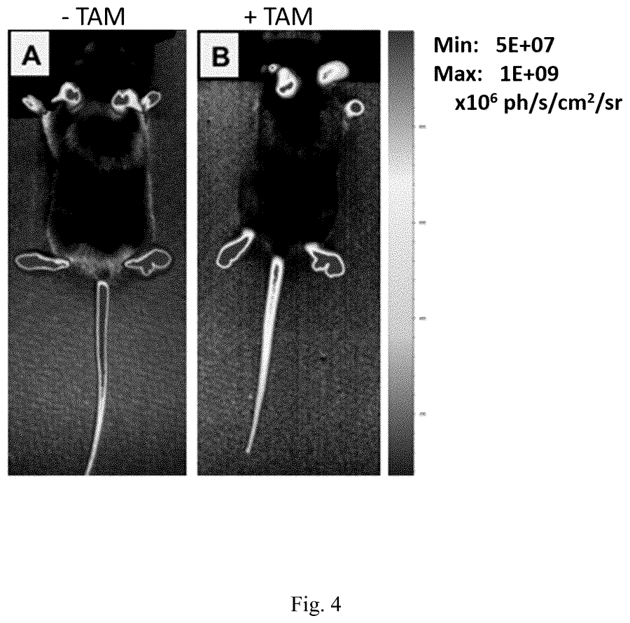

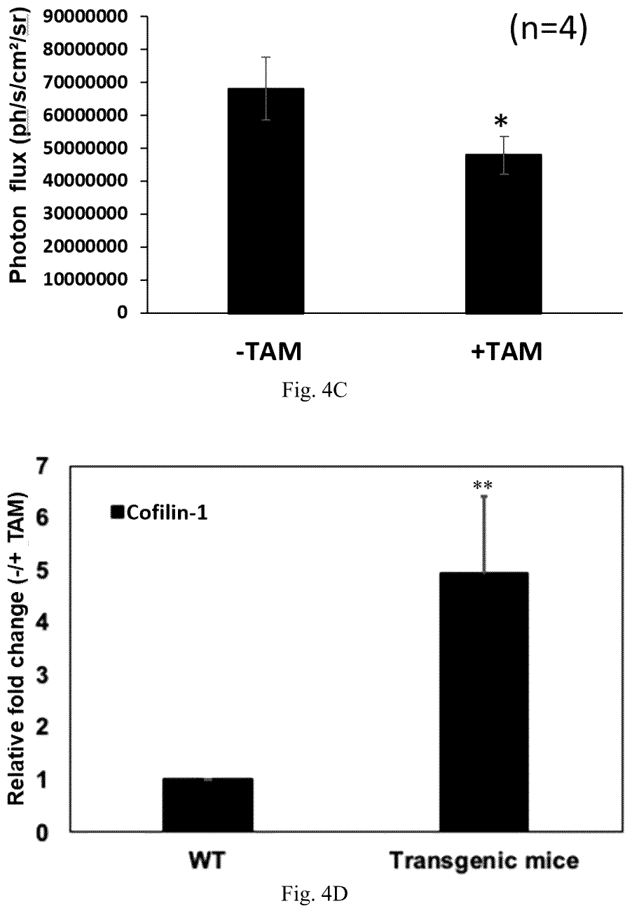

The expression of emGFP in transgenic mice was detected before and after the mouse was treated with tamoxifen (TAM). The Biospace Lab PhotonIMAGER Optima imaging system was used for detection of fluorescent signals in vivo. The whole-body photon flux was measured using the Biospace Lab M3 Vision software. The results showed that the GFP fluorescence was reduced after TAM treatment in transgenic mouse qualitatively (FIGS. 4, A and B) and quantitatively (FIG. 4C).

The expression of cofilin-1 gene in wild-type mice (WT) and loxP-emGFP-loxP/Cre-ERT2 transgenic mice was compared by quantitative PCR (qPCR) of RNA extracted from excised ear tissue. For qPCR, the primer set for cofilin-1 gene was: Forward: 5′-TGCCTGAGTGAGGACAAG-3′ (SEQ ID NO: 11) and Reverse 5′-GACAAAGGTGGCGTAGGG-3′ (SEQ ID NO: 12); and for S26 rRNA (internal control) was: Forward: 5′-CCGTGCCTCCAAGATGACAAAG-3′ (SEQ ID NO: 9) and Reverse: 5′-ACTCAGCTCCTTACATGGGCTT-3′ (SEQ ID NO: 10). The relative fold change of cofilin-1 mRNA was determined by mice with or without TAM treatment. The results showed that transgenic mice expressed about 5 folds of cofilin-1 mRNA up-regulation compared to WT mice (FIG. 4D). The result demonstrated the conditioned expression of transgenic cofilin-1 gene by TAM.

### Example 5

Immunohistochemical (IHC) Staining of Cofilin-1 Expression

The expression of cofilin-1 protein was determined by IHC method. In brief, the excised ear tissue was embedded in paraffin and then used for preparation of tissue slides. The slide was heated at 60° C. for 1 h, and the de-paraffined slide was rinsed by 95% and 75% ethanol twice, and then rinsed by phosphate buffered saline with Tween-20 (PBST). The slide was then placed in 10 mM citric acid buffer containing 0.1% Tween-20, pH 6.0 for heating at 121° C. for 4 min. The slide was then placed at room temperature for 20 min followed by treated with Dual Endogenous enzyme blocking solution at dark for 5 min. After rinsed with PBST, the sample was treated with goat serum for 30 min, and then replaced by anti-cofilin-1 antibody (1:100) at 4° C. overnight. After rinsed with PBST, the horseradish phosphate (HRP) conjugated secondary antibody was used for treatment for 20 min, rinsed, and covered by substrate working solution for 30 sec. The slide was then rinsed and treated with Mayer's hematoxylin for 30 sec then rinsed with ddH₂O, and mounted for microscopic examination.

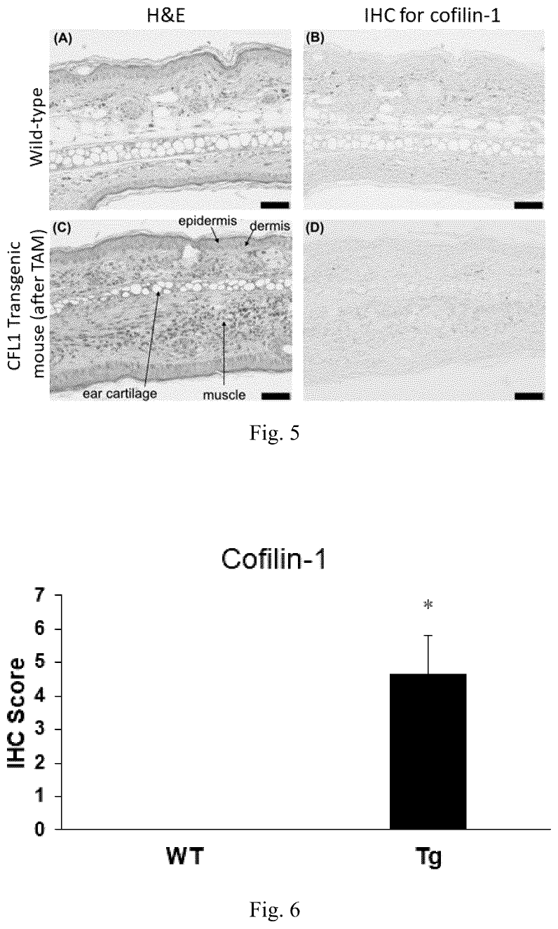

The results showed that cofiilin-1 protein was over-expressed in ear tissue of transgenic mouse treated with TAM compared to that of WT mouse (FIG. 5, A-D). The IHC staining was quantified by calculating the IHC score [proportion (1-4)×intensity (0-3)], and the results indicated that the cofilin-1 protein was widely and significantly expressed in ear tissues (FIG. 6).

### Example 6

The Body Weight Change in Cofilin-1 Transgenic Mouse Treated with TAM

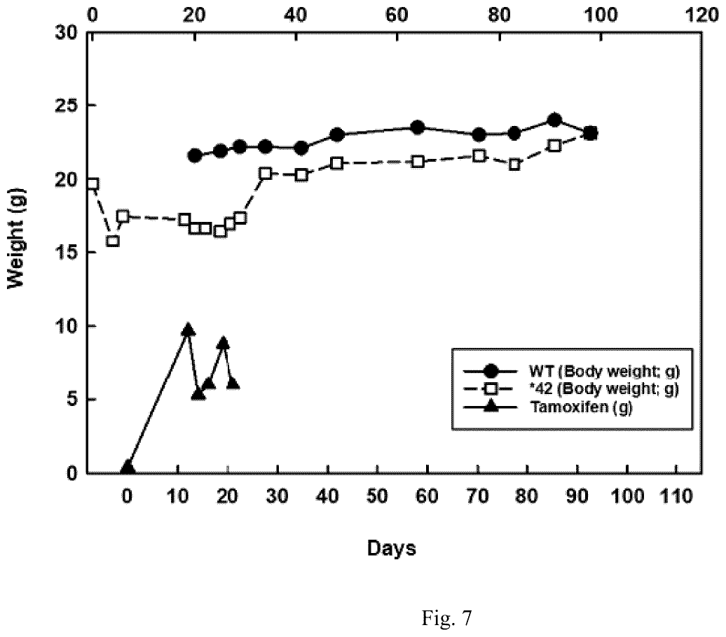

The mouse was fed with the TAM-containing feed for 21 days. The body weight of mouse was measured three times per week during feeding. The loss of TAM feed weight was also measured. It showed that transgenic mouse lost more body weight than wild-type mouse after fed with TAM feed (FIG. 7).

### Example 7

Effects of Cofilin-1 in the Moving Frequency of Transgenic Mice

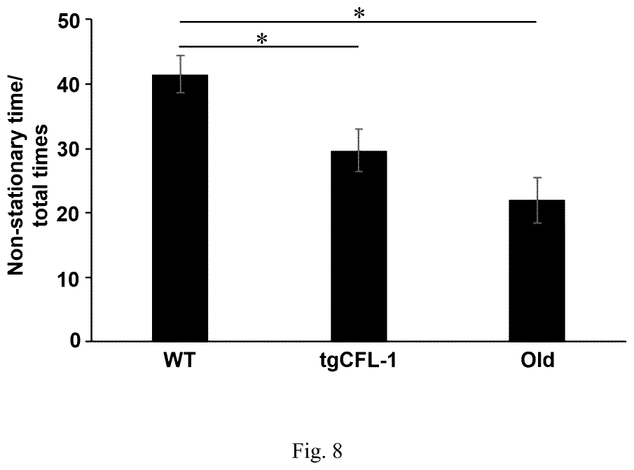

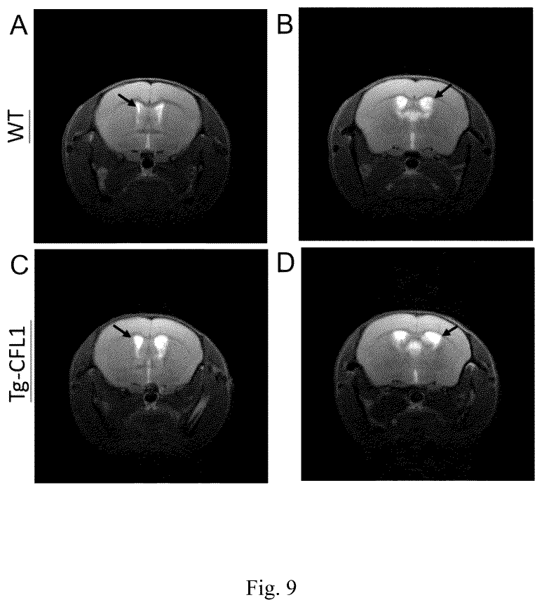

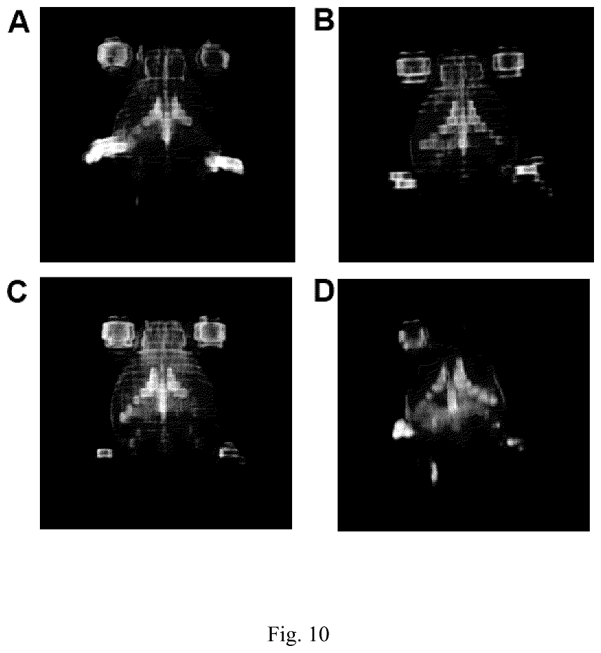

The behavior of cofilin-1 transgenic mouse was monitored and recorded by a web camera (Tapo C200, TP-LINK TAIWAN CO., LTD.) everyday up to one week. The camera will not record the mouse only if it started to move. The web camera will record the animal activity continuously until it took a rest or stop moving. Two web cameras were separately used for recording the activity of wild-type mouse and cofilin-1 transgenic mouse. The demonstration of mice activity monitored by web cameras was shown, and the infrared light would turn on for night recording of animal activity from 8 pm to 8 am when the light was turn off in the animal house. The moving frequencies of a young mouse (19 weeks), an old mouse (78 weeks), and a cofilin-1 transgenic mouse (19 weeks) were compared for 24 h. As mouse is nocturnal, it appears that the movement of young mouse at night was more frequently recorded than time that of old mouse and cofilin-1 transgenic mouse (FIG. 8). The results of video recorders were present here. Although the recordings were continuously performed for one week, different frequency of movement for the young mouse, the old mouse and the cofilin-1 transgenic mouse were similar during the period of recording.

### Example 8

Magnetic Resonance Imaging (MRI) Investigation of Brains in Cofilin-1 Transgenic Mice

A 7T microMRI was used for non-invasively imaging the brains of mice to explore the altered moving behaviors of cofilin-1 transgenic mice. The results showed that the lateral ventricle of cofilin-1 transgenic mice (Tg-CFL1) was larger than that of wild-type mouse at the similar slice of transverse images (FIG. 9, A-D). In addition, the 3D reconstruction of coronal imaging was performed to compare the difference between the lateral ventricles of wild-type mice and cofilin-1 transgenic mice (FIG. 10, A-D). Because the enlarged lateral ventricle is associated with aging, the MRI result implies that the age-like circadian rhythms exhibited in cofilin-1 transgenic mice is possibly related to altered lateral ventricle.

The foregoing detailed description and examples have been provided for clarity of understanding only. No unnecessary limitations are to be understood therefrom. The invention is not limited to the exact details shown and described; many variations will be apparent to one skilled in the art and are intended to be included within the invention defined by the claims.

What is claimed is:

1. A transgenic non-human multicellular organism model, the multicellular organism model being a progeny obtained from breeding first founders and second founders, wherein the first founders carry transgenic cfl1 gene controlled by loxP-fluorescent protein-loxP locus, and the second founders carry Cre-ERT2 transgenic gene.

2. The transgenic non-human multicellular organism model according to claim 1, wherein the progeny is treated with tamoxifen.

3. The transgenic non-human multicellular organism model according to claim 2, wherein the fluorescent protein is not expressed following the removal of the Stop sequence via tamoxifen.

4. The transgenic non-human multicellular organism model according to claim 3, wherein the fluorescent protein selected from the group consisting of a cyan fluorescent protein (CFP), a yellow fluorescent protein (YFP), a blue fluorescent protein (BFP), a green fluorescent protein (GFP), a red fluorescent protein (RFP) and an orange fluorescent protein (OFP).

5. A transgenic non-human multicellular organism model obtainable by a method comprising the following steps:
(1) a first founders carry transgenic cfl1 gene controlled by loxP-fluorescent protein-loxP locus;
(2) a second founders carry Cre-ERT2 transgenic gene;
(3) a progeny obtained from breeding first founders and second founders.

6. The method according to claim 5, wherein the progeny is treated with tamoxifen.

7. The method according to claim 6, wherein the fluorescent protein is not expressed following the removal of the Stop sequence via tamoxifen.

8. The method according to claim 6, wherein the fluorescent protein selected from the group consisting of a cyan fluorescent protein (CFP), a yellow fluorescent protein (YFP), a blue fluorescent protein (BFP), a green fluorescent protein (GFP), a red fluorescent protein (RFP) and an orange fluorescent protein (OFP).


SEQUENCE LISTING (12 sequences)

SEQ ID NO: 1 - DNA, 23 bp, synthetic construct
  source 1..23: mol_type = other DNA
ctaaacatgcttcatcgtcggtc

SEQ ID NO: 2 - DNA, 23 bp, synthetic construct
  source 1..23: mol_type = other DNA
tctgaccagagtcatccttagcg

SEQ ID NO: 3 - DNA, 23 bp, synthetic construct
  source 1..23: mol_type = other DNA
ccgaaagttgccttttatggctc

SEQ ID NO: 4 - DNA, 24 bp, synthetic construct
  source 1..24: mol_type = other DNA
gctgcaaagagtctacacgctagg

SEQ ID NO: 5 - DNA, 23 bp, synthetic construct
  source 1..23: mol_type = other DNA
atggcctccggtgtgtggctgtc

SEQ ID NO: 6 - DNA, 19 bp, synthetic construct
  source 1..19: mol_type = other DNA
tcacaaaggcttgccctcc

SEQ ID NO: 7 - DNA, 21 bp, synthetic construct
  source 1..21: mol_type = other DNA
gctgtggcgtcctataaaacc

SEQ ID NO: 8 - DNA, 23 bp, synthetic construct
  source 1..23: mol_type = other DNA
gaccctgcagtgaggataacttc

SEQ ID NO: 9 - DNA, 22 bp, synthetic construct
  source 1..22: mol_type = other DNA
ccgtgcctccaagatgacaaag

SEQ ID NO: 10 - DNA, 22 bp, synthetic construct
  source 1..22: mol_type = other DNA
actcagctccttacatgggctt

SEQ ID NO: 11 - DNA, 18 bp, synthetic construct
  source 1..18: mol_type = other DNA
tgcctgagtgaggacaag

SEQ ID NO: 12 - DNA, 18 bp, synthetic construct
  source 1..18: mol_type = other DNA
gacaaaggtggcgtaggg